(19) United States
(12) Patent Application Publication
(10) Pub. No.: US 2013/0289725 A1
(43) Pub. Date: Oct. 31, 2013

(54) System and Device for Providing Nutrition to Intervertebral Tissue and Method of Use

(71) Applicants: Brian Donovan, San Jose, CA (US); Gregory Arcenio, Redwood City, CA (US); Erika Palmer, Menlo Park, CA (US)

(72) Inventors: Brian Donovan, San Jose, CA (US); Gregory Arcenio, Redwood City, CA (US); Erika Palmer, Menlo Park, CA (US)

(73) Assignee: KYPHON SARL, Neuchatel (CH)

(21) Appl. No.: 13/455,638

(22) Filed: Apr. 25, 2012

(51) Int. Cl. A61F 2/44; A61B 17/88; A61F 2/28

(52) U.S. Cl. 623/17.12; 623/16.11; 606/80

(57) ABSTRACT

A bone implant device includes a barrel having a sharpened distal end for advancing into bone and a proximal end portion configured to engage a driver end for advancing and turning the barrel to implant the barrel into bone. A channel is formed longitudinally through at least a portion of the barrel and is open ended on the proximal end portion. A permeable material is disposed within the channel that permits fluid communication therethrough between the distal end and the channel. Methods of use are disclosed.

### TECHNICAL FIELD

The present disclosure generally relates to medical devices for the treatment of musculoskeletal disorders, and more particularly to a surgical system, device and method for supplying nutrition to disc tissues between vertebrae.

### BACKGROUND

Spinal pathologies and disorders such as scoliosis and other curvature abnormalities, kyphosis, degenerative disc disease (DDD), disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. In some cases, the spinal disorder is linked to lack of nutrition of intervertebral discs.

Since it has been shown that disc nutrition is directly linked to the health of the disc, a reduction of the nutrient flow can lead to deterioration. Furthermore, as maturity occurs, a network of blood vessels in an annulus fibrosus of the disc does not usually penetrate beyond two or three lamellae and endplates on the vertebral body in contact with the disc undergo extensive mineralization and restrict blood flow and nutrient delivery to the disc. As these conditions persist, the likelihood of disc degeneration increases. This disclosure describes improvements in promoting disc health.

### SUMMARY

Accordingly, a system, device and method for providing nutrients to a disc of a spine are provided. In one embodiment, in accordance with the principles of the present disclosure, a bone implant device is provided. The bone implant device includes a barrel having a sharpened distal end for advancing into bone and a proximal end portion configured to engage a driver end for advancing and turning the barrel to implant the barrel into bone. A channel is formed longitudinally through at least a portion of the barrel and is open ended on the proximal end portion. A permeable material is disposed within the channel that permits fluid communication therethrough between the distal end and the channel.

In one embodiment, a bone implant system includes an implant device including a barrel having a sharpened distal end for advancing into bone, and a proximal end portion. A channel is formed longitudinally through at least a portion of the barrel and is open ended on the proximal end portion. A permeable material is disposed within the channel that permits fluid communication therethrough between the distal end and the channel. A drill device includes the implant device loaded therein for delivery to and implantation at a surgical site. The drill device includes a driver end detachably coupled to the implant device wherein features on the proximal end portion of the implant device fit into the driver end to permit for advancing motion and rotation for driving the implant device into bone. A length of cable has a wire therein coupled to the driver end, a knob is coupled to the wire for controlling rotation of the driver end when the knob is advanced and turned and an articulating tip portion is provided on a distal end of the cable. The articulating tip portion has a tip capable of articulating to a perpendicular configuration relative to the length of cable. The tip includes a housing for enclosing the implant device for deployment to a surgical site. A handle arm is provided wherein activating the handle arm controls the articulation of the tip portion by advancing a push rod through the cable such that by activating the handle arm, the push rod rotates and limits the articulation of the tip portion. The drill device implants the implant device through a vertebral body wall and through an end plate to provide nutrients to an intervertebral disc.

In one embodiment, a method for providing nutrients to an intervertebral disc is provided. The method comprises the steps of: loading a bone implant device in a tip portion of a drill device in contact with a driver end, the bone implant device including a barrel having a sharpened distal end for advancing into bone tissue, and a proximal end portion configured to engage the driver end for advancing and turning the barrel to implant the barrel into bone, a channel formed longitudinally through at least a portion of the barrel and being open ended on the proximal end portion; and a permeable material disposed within the channel that permits fluid communication therethrough between the distal end and the channel; providing access to an interior surface of a vertebral body adjacent to an intervertebral disc; articulating the tip portion of the drill device to expose the interior surface to a surface of the tip portion; and implanting the implant device through a wall of the vertebral body and through an adjacent endplate to provide nutrients through the implant device to the intervertebral disc.

### BRIEF DESCRIPTION OF THE DRAWINGS

The present disclosure will become more readily apparent from the specific description accompanied by the following drawings, in which:

FIG. 1 is an illustrative view of an intervertebral disc and adjacent vertebral bodies showing implanted tubes installed through an endplate to provide nutrients to the disc in accordance with the principles of the present disclosure;

FIG. 2 is a perspective view of an illustrative implant in accordance with one embodiment;

FIG. 3 is a perspective view of another illustrative implant having a thread for driving the implant into bone in accordance with one embodiment;

FIGS. 4A-4F show cross-sectional views of implant devices in different configurations in accordance with illustrative embodiments;

FIG. 5 is a side view of a drill device having a side panel removed to show internal components of the device in accordance with one embodiment;

FIG. 6 is a magnified view of a tip portion of the drill device of FIG. 5 in an articulated configuration and shown in phantom to visualize internal components in accordance with one embodiment;

FIG. 7 is a magnified view of the tip portion of the drill device of FIG. 5 in an articulated configuration in accordance with one embodiment; and

FIG. 8 is a magnified view of a tip portion of the drill device of FIG. 5 with a tip housing removed to view the driver end and a loaded implant device in a neutral non-articulated configuration in accordance with one embodiment;

Like reference numerals indicate similar parts throughout the figures.

### DETAILED DESCRIPTION

The exemplary embodiments of the surgical system, devices and related methods of use disclosed are discussed in terms of medical devices for the treatment of musculoskeletal disorders and more particularly, in terms of a surgical system and method for treatment of a spine disorder. It is envisioned that the surgical system and method may be employed in applications such as treatment of diseases or disorders, such as degenerative disc disease, etc. For example, the surgical system and method can include a drill system configured to place hollow tubes through a vertebral end plate to improve nutrition of intervertebral disc tissue.

In one embodiment, the system and method include a device that provides a way of restoring nutrition to the disc by providing a passageway for nutrients across the endplates. Disc nutrition has been shown to be directly linked to the health of the disc, and a reduction in nutrients can lead to deterioration. Through the vertebral body, a tool can be used to pierce the endplate and leave a device behind that has a channel and/or micro-perforations to allow for controlled nutrient flow from the vertebral body. The present principles provide the ability to restore disc nutrition after endplates have calcified and cutoff or reduced nutrient flow to the disc. The device left in the vertebral body should prevent further disc degeneration and possibly prevent the need for nucleus/disc replacement or the fusion of the vertebrae.

In one embodiment, nutrient flow is restored or increased to the intervertebral disc through endplate nutrition. Nutrient transport across the endplates can be achieved by having a drill tip perforated with small holes on the order of, e.g., about 25 to about 50 microns, drilling in the tip or bit and then leaving the drill bit behind. A hollow tube could also achieve similar results, especially if it were to be filled with a porous material that controlled nutrient transport rates.

The need for disc nutrition addresses different deficiencies and deteriorations of the spinal column. The disclosed embodiments permit for a minimal size instrument/implant to be loaded on a drill device with an ability to angle a head of the drill device to permit a surgeon the ability to manipulate the angle to permit drilling from in between two vertebral bodies.

It is contemplated that one or all of the components of the surgical system may be disposable, peel-pack, pre-packed sterile devices. One or all of the components of the surgical system may be reusable. The surgical system may be configured as a kit with multiple sized and configured components.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system and methods may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column, as well as the repair and treatment of other joints, such as the knee, hip, elbow, shoulder, etc. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

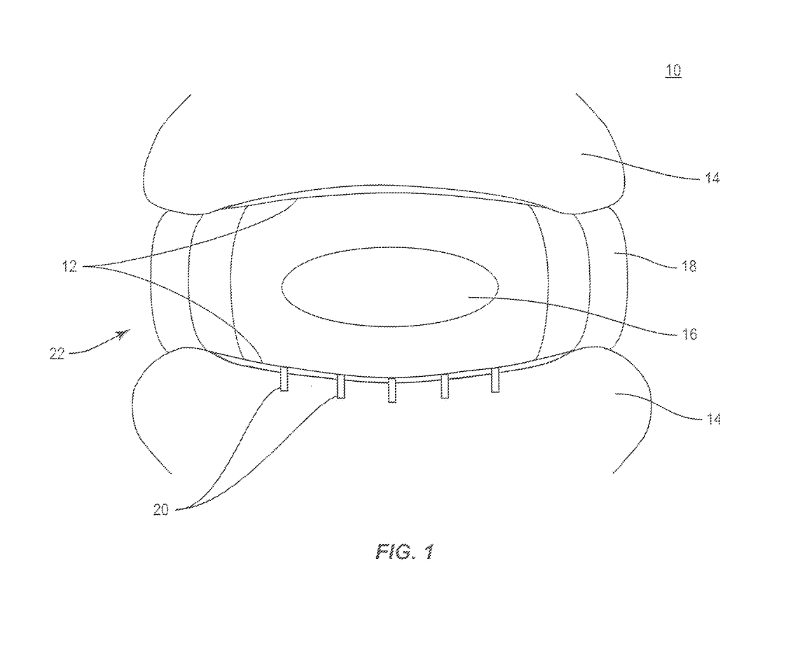

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-8, there are illustrated components of a surgical system, such as, for example, implantable tubes or drill bits, a drill configured to implant the tubes or drill bits and related methods of use in accordance with the principles of the present disclosure.

The components of the implantable tube or drill bits and the drill configured to implant the tubes or drill bits can be fabricated from biologically acceptable materials suitable for medical applications, including metals, synthetic polymers, ceramics and bone material and/or their composites, depending on the particular application and/or preference of a medical practitioner. For example, the components, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of correction system 10 may have material composites, including the above materials, to achieve various desired characteristics such as strength, rigidity, elasticity, compliance, biomechanical performance, durability and radiolucency or imaging preference. The components, individually or collectively, may also be fabricated from a heterogeneous material such as a combination of two or more of the above-described materials. The components may be monolithically formed, integrally connected or include fastening elements and/or instruments, as described herein.

Referring to FIG. 1, is a schematic diagram shows an intervertebral disc region 10. The region 10 includes a disc 22 disposed between two vertebral bodies 14. The disc 22 includes an annulus fibrosus surrounding a nucleus pulpous 16. End plates 12 are disposed between the disc 22 and the vertebral bodies 14. As described above, blood vessels in the annulus fibrosus 18 of the disc 22 may undergo extensive mineralization and become restrictive of blood flow and nutrient delivery to the disc 22. As these conditions persist, the likelihood of disc degeneration increases.

In accordance with the present principles, leave-behind devices 20 are installed through the end plate 12 and into bone tissue or the vertebral body 14. Devices 20 may include drill bits and may includea plurality of different forms including tubes, tubes with threads, perforated tubes, tubes filled with porous material to control nutrient flow, tubes having a membrane therein to control nutrient flow, etc. Devices 20 may be installed, for example, with an open, mini-open or minimally invasive surgical technique to implant the devices 20 in vertebrae of a selected section of the spine.

This approach is useful in a general degeneration scenario for the disc 22 but also to heal fissures or tears in the annulus 18. The outer portions of the annulus 18 can more easily heal and scar from traumatic damage. However, a restoration of nutrients could help inner portions of the disc 22 in this healing process as well.

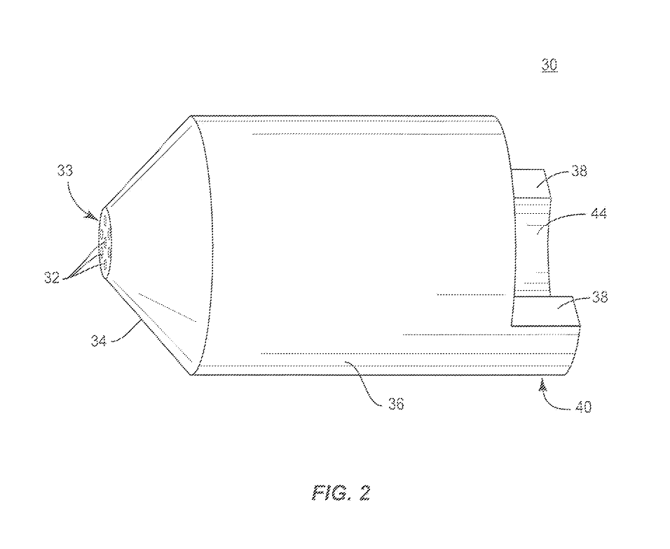

Referring to FIG. 2, a perspective view of a leave-behind device 30 (similar to devices 20 of FIG. 1) is shown in accordance with an illustrative embodiment. Device 30 includes a distal end portion 34 which is sharpened or inclined toward a perforated tip 33 having a plurality of holes 32 formed therein. The holes 32 in the tip 33 pass through an entire length of a barrel 36 of the device 30. The holes 32 provide redundant paths of a particular cross-sectional dimension to control the flow of nutrients through the device 30. In alternate embodiments, a center channel in the barrel 36 is filled with a permeable material that permits the flow of nutrients and controls the rate of flow by selecting an appropriate permeability/porosity.

Barrel 36 illustratively includes a smooth surface; however, other surface finishes may be employed depending on the application and desired results. For example, the surface of the barrel 36 and the distal end portion 34 may include one or more different surface finishes or geometric features. The surface finishes may include a roughed surface, a gnarled surface, a grooved surface, surface adhesive, etc. Such surface may assist in anchoring the device 30 in bone tissue.

A proximal end portion 40 of the device 30 includes an interface for a drilling tool. The device 30 needs to be gripped and turned to be installed and then needs to be easily detached to leave the device 30 behind in bone tissue. Proximal end portion 40 includes an arcuate protrusion 44 that include flats 38 that interface with a driver end or a drill device or tool. The arcuate protrusion 44 provides axial stability while the flats 38 provide surfaces against which torque can be applied for turning the device 30 in a drilling operation. Since the device 30 is smooth, a forward pressure provided by the drilling tool may be needed to drive the device into the endplate (12, FIG. 1).

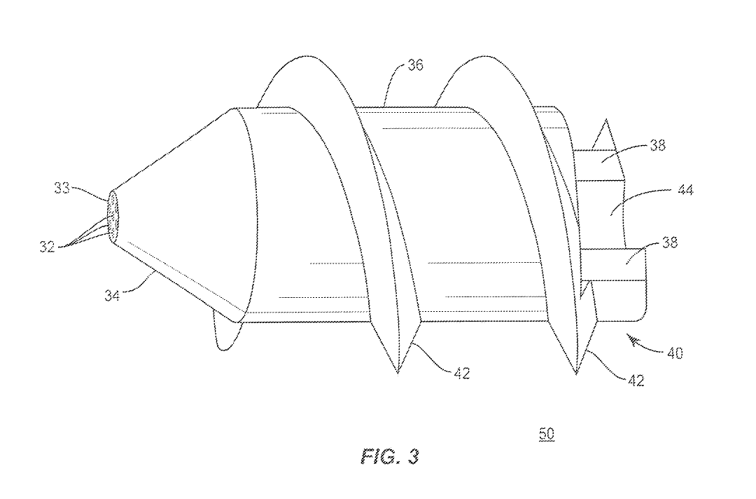

Referring to FIG. 3, a leave-behind device 50 is shown which is similar to the device 30 of FIG. 2. Device 50 includes threads 42 that may help in driving the device 50 deeper into the endplate (12, FIG. 1). In the embodiment shown, the barrel 36 is a base diameter on which the threads 42 are formed. In use, once the threads 42 are engaged during the drilling process, the device 50 will be driven in by the threads 42 rather than a forward pressure provided by the drilling tool. In addition, the threads 42 will enhance the stability of the device 50 to ensure the device 50 remains in place.

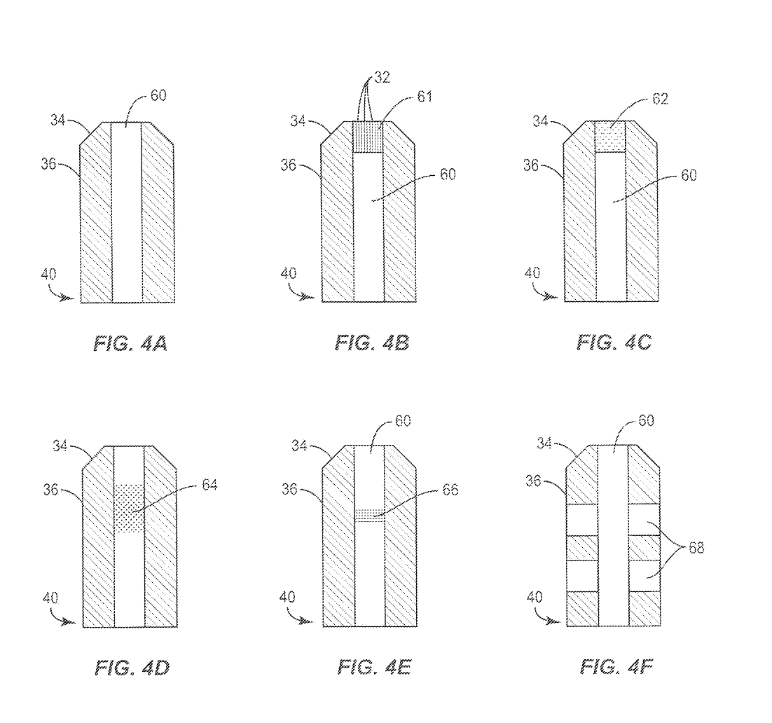

Referring to FIGS. 4A-4F, different configurations of the device 30 are illustratively shown. Note that each of the embodiments shown in FIGS. 4A-4F may include threads 42 described with reference to FIG. 3. The embodiments of FIGS. 4A-4F are shown without drill engagement features for simplicity. In FIG. 4A, the device 30 is shown having a central channel 60 extending an entire length of the device 30. The distal end portion 34 is sharpened to permit penetration into an end plate. This configuration permits a large flow of nutriments since the central channel 60 is open. This configuration needs to be inspected to ensure that the channel 60 does not clog during installation. In one embodiment, a diameter of the channel 60 is about 0.25 mm, a diameter of the barrel 36 is about 0.5 mm and a length of the device 30 is about 3 mm. Other dimensions are also contemplated.

In FIG. 4B, the device 30 includes a structure 61 having a plurality of through holes 32 formed therein. The holes 32 are open between the sharpened distal end and the channel 60. The holes 32 may have a diameter of between about 25 microns to about 50 microns, although other sizes may be employed. The structure 61 is preferably integrally formed with the device 30. The holes 32 may be formed by laser drilling, etching or other processes. The structure 61 improves drilling performance. The holes 32 provide a reduced cross-sectional area from the channel 60 and permit the flow rate and particle size to be selected and controlled.

In FIG. 4C, the device 30 includes a medication 62, which may include an anesthetic, anti-inflammatory or other medication. The medication 62 is configured to dissolve during the recovery process and eventually leave channel 60 open for nutrient flow. The medication 62 may be configured to line the channel 60 but also permit nutrient flow. The medication 62 may be applied externally to the device as well. In one example, an anesthetic is employed which may be slowly released into the disc to relieve pain while disc healing occurs.

In FIG. 4D, the device 30 includes a porous material 64 to control the nutrient flow rate. The porous material 64 may include any medical grade porous material that permits a flow rate therethrough. The pores provide a reduced cross-sectional area from the channel 60 and permit the flow rate and particle size to be selected and controlled.

In FIG. 4E, the device 30 includes a permeable membrane 66 to control the nutrient flow rate. The membrane 66 may include any medical grade synthetic or natural membrane material that permits a flow rate therethrough. The membrane 66 provides a reduced flow through the channel 60 and permits the flow rate and particle size to be selected and controlled. In FIGS. 4D and 4E, the porous material 64 and/or the membrane 66 may be packed in the channel 60 or attached in the channel 60 by a mechanical means (e.g., clamped, inserted in a slot, etc.) or by chemical means (e.g., an adhesive, etc.).

In FIG. 4F, the device 30 is shown having perforation holes 68 to reduce clogging of the channel 60 upon installation and provides a greater surface area for possible nutrient flow while reduces clogging. It should be understood that the embodiments shown in FIGS. 4A-4F may be combined in any manner. For example, the structure 61 of FIG. 4B may be combined with the holes of FIG. 4F. Likewise, the medication 62 of FIG. 4C may be combined with membrane 66 of FIG. 4E. Other combinations are contemplated. It should also be understood that the examples provided in FIGS. 4A-4F are illustrative and non-limiting as other configurations and features may be provided in accordance with the present principles. After application of the device 30, a bone fill might be necessary to fill any voids created by the drilling.

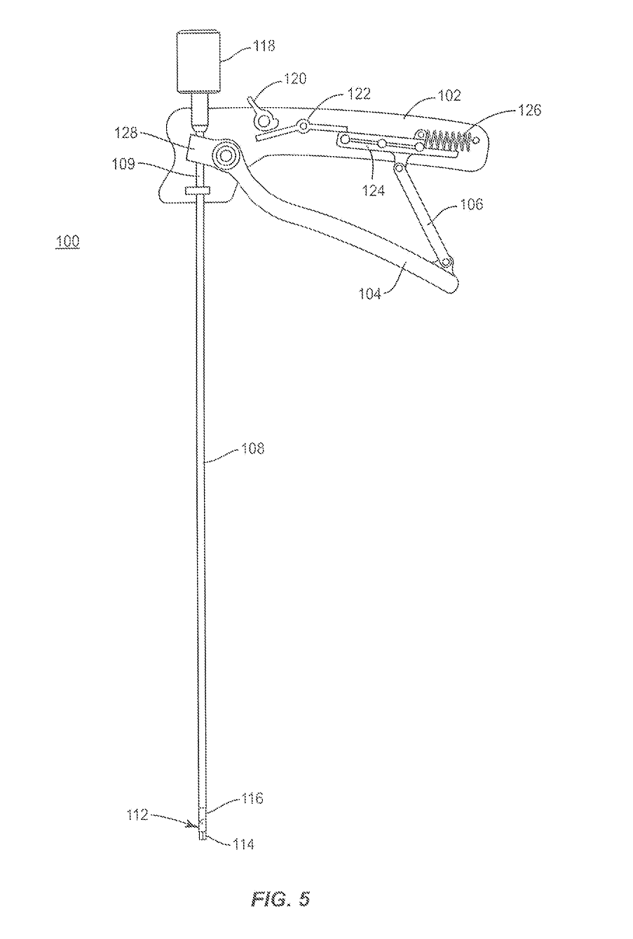

Referring to FIG. 5, an illustrative drilling device 100 is shown in accordance with one embodiment. Access to the endplates is gained through a vertebral body. A working cannula (not shown) is passed into the vertebral body. The device 100 includes a cable 108 that passes into the vertebral body through the cannula. The cable 108 includes a unidirectional torque drive cable or a Nitinol wire 109 is disposed within a sheath of the cable 108. The wire connects with an adaptor ring or end part 116 of an angularly adjustable tip portion 112. The tip portion 112 includes a tip 114 adapted to receive a leave-behind device, such as, those described with reference to FIGS. 1-4.

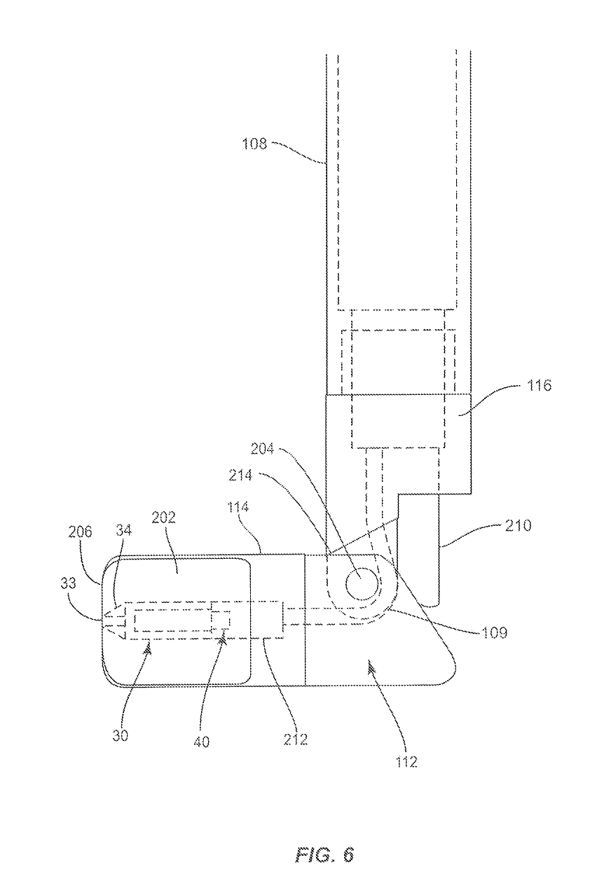

The device 100 includes a handle portion 102. The handle portion 102 supports a handle arm 104, which is pivotally connected to a link 106. The link 106 pivotally connects to a slider link 124, which is biased by a spring 126. The spring 126 draws the slider link 124 proximally to maintain the handle arm 104 in an extended position. Squeezing the handle arm 104 articulates the tip portion 112 by up to 90° to a ready-to-drill position by advancing a push rod (FIG. 6) connected to an end portion 128 of the handle arm 104 toward the tip portion 112. The push rod at the tip portion 112 causes the tip 114 to bend about a hinged connection. A knob 118 provides rotation of the tip 114 and can also exert forward pressure on the tip 114 by permitting additional forward motion of the wire 109 during drilling. The handle portion 102 may include other features, such as a switch 120 employed to activate a locking mechanism 122. The locking mechanism 122 is employed to lock the slider link 124 to lock the angle of articulation by locking the handle arm 104. Other features may be included on the device 100.

Referring to FIG. 6, a magnified view of the adjustable tip portion 112 of the device 100 is shown in greater detail. The magnified view is shown in phantom so that internal components are visible. FIG. 6 shows the tip portion 112 in an articulated position. Positive engagement of a push rod 210 prevents overdriving of the tip 114 beyond 90°. When the tip 114 reaches 90° relative to an end part 116 by pivoting on a dowel or pin 204, the push rod 210 prevents driving the tip 114 any further. The push rod 210 limits the further rotation of the tip 114 with respect to end part 116 which may engage a portion 214 of end part 116. The angle of articulation may be adjusted between 0 and 90 degrees and locked in place if needed. A driver end 212 interfaces with the proximal end portion 40 of the leave-behind device 30. The driver end 212 is attached to the end of the wire 109. The device 30 is shown in cross-section to identify its features. The device 30 is shown in a loaded position within a cavity 202 formed in the tip 114.

In operation, a surface 206 of the tip 114 is brought in contact with a surface of bone tissue where device 30 is to be driven in. By turning the cable 109 (and applying a forward pressure, if needed), the driver end 212 turns and pushes the device 30 into the bone tissue piercing the end plates into the disc as described above. The tip 114 provides a housing that stabilizes the device 30 during drilling.

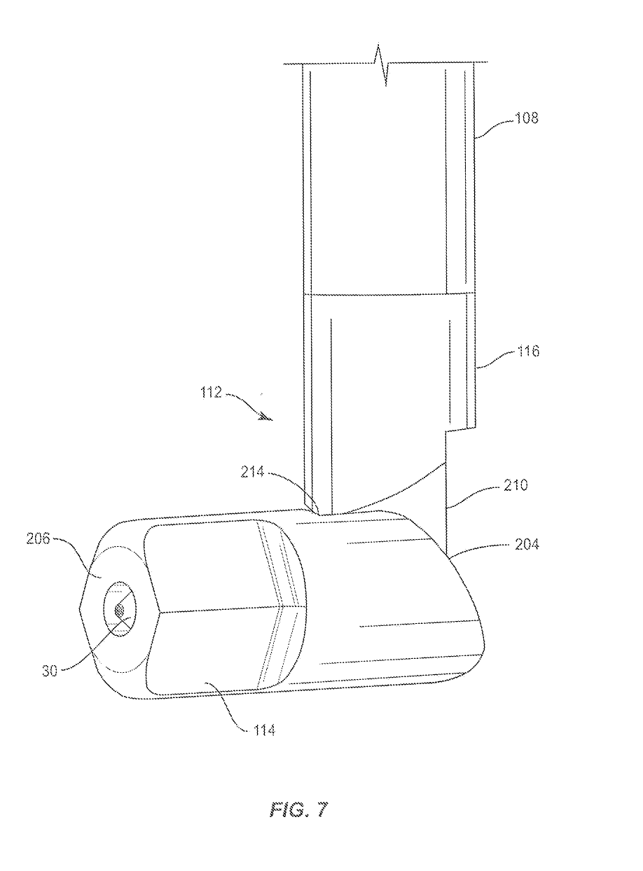

Referring to FIG. 7, a perspective view of the tip portion 112 is shown. The cable 108 includes an outer sheath that is terminated in the end part 116. The push rod 210 is shown engaging the tip 114. The tip 114 houses a drive train (wire 109 with the driver end 212, which are hidden in FIG. 7). The device 30 is also partially hidden in the housing of the tip 114. The device 30 is protected and guided by the housing of the tip 114. When the drill is ready to be engaged, the tip 114 is locked at, e.g., 90° by advancing (and locking) the push rod 210, and the drill is ready to extend and turn by further advancing and turning the wire 109.

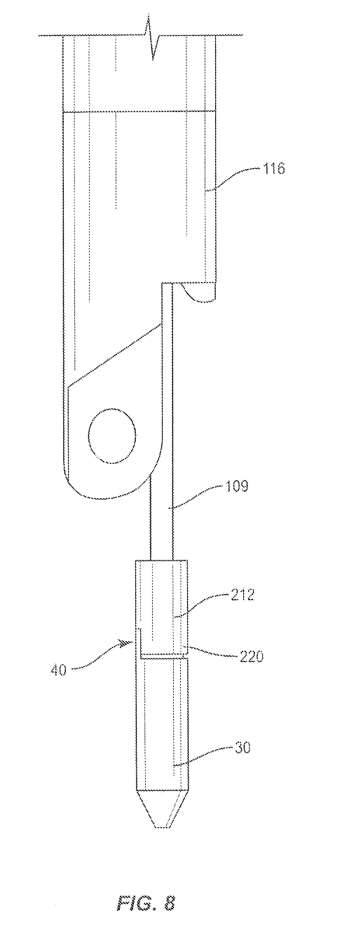

Referring to FIG. 8, the device 30 is located in a neutral position and not positioned for drilling. The tip 114 is removed to show the device 30 engaged with driver end 212. The driver end 212 includes features 220 that are configured to engage the proximal end portion 40 of the device 30. The features 220 fit into the device 30 to be capable of imparting rotation and axial movement to the device 30 to cause the device 30 to be driven into bone tissue. The device 30 may remain engaged with the driver end 212 by providing a force therebetween, using the housing of the tip 114 to secure the device 30, using an adhesive, magnetics or other contact forces that permit drilling and subsequent detachment from the driver end 212.

In assembly, operation and use, devices 30 and 100 would be employed together as described above, in a surgical procedure, such as, for a treatment to treat disc disorders including but not limited to degenerative disc disease. It is contemplated that one or all of the components of the system can be delivered or implanted as a pre-assembled device or can be assembled in situ. Components of the system may be completely or partially revised, removed or replaced.

For example, as shown in FIGS. 1-8, devices 30, described above, can be employed with a surgical treatment of an applicable condition or injury of an affected section of a spinal column and adjacent areas within a body, such as, for example, vertebrae. It is envisioned that devices 30 may be employed with one or a plurality of vertebrae.

In use, to treat vertebrae, a medical practitioner obtains access to a surgical site including vertebrae in any appropriate manner, such as through incision and retraction of tissues. It is envisioned that devices 30 can be used in any existing surgical method or technique including open surgery, mini-open surgery, minimally invasive surgery and percutaneous surgical implantation, whereby a vertebrae is accessed through a mini-incision, sleeve or cannula that provides a protected passageway to the area. Once access to the surgical site is obtained, the particular surgical procedure can be performed for treating the spine disorder. The number, size and location of the devices 30 to be implanted may be determined in advance of the surgical procedure.

An incision is made in the body of a patient and a cutting instrument (not shown) creates a surgical pathway for implantation of components such as device 100 for implanting devices 30. A preparation instrument (not shown) can be employed to prepare tissue surfaces of vertebrae, as well as for aspiration and irrigation of a surgical region according to the requirements of a particular surgical application.

An opening or openings are made in selected vertebra or vertebrae for receiving the device 100 therein. Device 100 is then loaded with a device 30 and inserted to a selected position through the surgical pathway. Once in position, the handle arm 104 is squeezed to articulate the tip position 112 by an angle. The tip 114 is placed at a drill site and the device 30 is advanced and turned using the device 100 to implant the device 30 through the end plates of the vertebral body, according to the particular requirements of the surgical treatment. These steps are repeated as necessary.

In one embodiment, an agent may be delivered to the surgical site, which may be disposed, packed or layered within, on or about the components and/or surfaces of the device 30. It is envisioned that the agent may include bone growth promoting material, such as, for example, bone graft to enhance fixation of the fixation elements with vertebrae or other medication.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of correction system 10 can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair spinal deterioration or damage, with the aid of the present principles. Upon completion of the procedure, the surgical instruments and assemblies are removed and the incision is closed.

It is contemplated that the devices 30 may be employed to treat any spinal disease or injury by promoting nutrient flow into the intervertebral disc. It is further contemplated that the components and methods of use may be used to prevent or minimize future injury.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone implant device comprising:
a barrel having a sharpened distal end for advancing into bone, and a proximal end portion configured to engage a driver end for advancing and turning the barrel to implant the barrel into bone;
a channel formed longitudinally through at least a portion of the barrel and being open ended on the proximal end portion; and
a permeable material disposed within the channel that permits fluid communication therethrough between the distal end and the channel.

2. A bone implant device as recited in claim 1, wherein the implant device includes a thread spiral about a periphery for driving the implant device into bone.

3. A bone implant device as recited in claim 1, wherein the implant device includes transverse holes communicating with the channel.

4. A bone implant device as recited in claim 1, wherein the distal end includes a tip formed in the channel, the tip including a plurality of longitudinally disposed holes communicating with the channel, the holes having a size less than a size of the channel.

5. A bone implant device as recited in claim 4, wherein holes have a diameter of between about 25 microns and about 50 microns.

6. A bone implant device as recited in claim 1, wherein the permeable material includes a porous solid disposed within the channel.

7. A bone implant device as recited in claim 1, wherein the permeable material includes a membrane disposed within the channel.

8. A bone implant device as recited in claim 1, further comprising medication disposed in or on the implant device.

9. A bone implant device as recited in claim 1, further comprising a driver end detachably coupled to the implant device wherein features on the proximal end portion of the implant device fit into the driver end to permit for advancing motion and rotation for driving the implant device into bone.

10. A bone implant device as recited in claim 9, wherein the driver end is coupled to a wire that is advanced and turned by a drill device.

11. A bone implant device as recited in claim 10, wherein the drill device includes a length of cable having the wire therein and an articulating tip portion on a distal end of the cable, the articulating tip portion having a tip capable of articulating to an angled configuration relative to the length of cable.

12. A bone implant device as recited in claim 11, wherein the tip includes a housing for enclosing the implant device for deployment to a surgical site.

13. A bone implant device as recited in claim 12, wherein the tip includes a surface for contacting bone and the implant device is extended beyond the surface when being implanted in the bone.

14. A bone implant device as recited in claim 11, wherein the drill device includes a handle arm configured so that when the handle arm is activated it controls the articulation of the tip portion.

15. A bone implant device as recited in claim 14, wherein the handle arm connects to push rod configured to advance through a cable such that by activating the handle arm, the push rod rotates and limits the articulation of the tip portion.

16. A bone implant device as recited in claim 10, wherein the drill device includes a knob coupled to the wire for controlling rotation of the driver end.

17. A bone implant device as recited in claim 1, wherein the sharpened distal end is configured to bore through a vertebral body wall and through an end plate to provide nutrients to an intervertebral disc.

18. A bone implant system comprising:
an implant device including:
a barrel having a sharpened distal end for advancing into bone and a proximal end portion;
a channel formed longitudinally through at least a portion of the barrel and being open ended on the proximal end portion; and
a permeable material disposed within the channel that permits fluid communication therethrough between the distal end and the channel; and
a drill device having the implant device loaded therein for delivery to and implantation at a surgical site, the drill device including:
a driver end detachably coupled to the implant device wherein features on the proximal end portion of the implant device fit into the driver end to permit advancing motion and rotation for driving the implant device into bone;
a length of cable having a wire therein coupled to the driver end;
a knob coupled to the wire for controlling rotation of the driver end when the knob is advanced and turned;
an articulating tip portion on a distal end of the cable, the articulating tip portion having a tip capable of articulating up to a perpendicular configuration relative to the length of cable, the tip including a housing for enclosing the implant device for deployment to a surgical site; and
a handle arm wherein activating the handle arm controls the articulation of the tip portion by advancing a push rod through the cable such that by activating the handle arm, the push rod rotates and limits the articulation of the tip portion wherein the drill device implants the implant device through a vertebral body wall and through an end plate to provide nutrients to an intervertebral disc.

19. A method for providing nutrients to an intervertebral disc, the method comprising the steps of:
loading a bone implant device in a tip portion of a drill device in contact with a driver end, the bone implant device including a barrel having a sharpened distal end for advancing into bone, and a proximal end portion configured to engage the driver end for advancing and turning the barrel to implant the barrel into bone, a channel formed longitudinally through at least a portion of the barrel and being open ended on the proximal end portion; and a permeable material disposed within the channel that permits fluid communication therethrough between the distal end and the channel;
providing access to an interior surface of a vertebral body adjacent to an intervertebral disc;
articulating the tip portion of the drill device to expose the interior surface to a surface of the tip portion; and
implanting the implant device through a wall of the vertebral body and through an adjacent endplate to provide nutrients through the implant device to the intervertebral disc.

20. A method for providing nutrients to an intervertebral disc as recited in claim 19, further comprising controlling nutrient flow in accordance with a permeability of the permeable material.
